(19) United States
(12) Patent Application Publication
(10) Pub. No.: US 2024/0315666 A1
(43) Pub. Date: Sep. 26, 2024

(54) SYSTEM AND METHOD FOR IDENTIFYING A TARGET PROBE FROM A PLURALITY OF PROBES COLLABORATING WITH AN EXAMINATION DEVICE

(71) Applicant: SUPERSONIC IMAGINE, AIX EN PROVENCE (FR)

(72) Inventors: Etienne ROUSSEAU, AIX EN PROVENCE (FR); Hélène SPORTOUCHE, MARSEILLE (FR)

(21) Appl. No.: 18/546,060

(22) PCT Filed: Feb. 1, 2022
(86) PCT No.: PCT/EP2022/052309

§ 371 Date: Aug. 10, 2023

(30) Foreign Application Priority Data
Feb. 17, 2021 (FR) FR2101522

(51) Int. Cl. A61B 8/00; A61B 90/00; A61B 90/98

(52) U.S. Cl. CPC A61B 8/4438; A61B 90/36; A61B 90/98; A61B 2090/0807

(57) ABSTRACT

A system and method for identifying a target probe from a plurality of probes collaborating with an examination device, where the system includes an identification element configured to be associated with the target probe, at least one probe-holder configured to be associated with the examination device and to support the target probe, a reader associated with the probe-holder and configured to communicate with the identification element of the target probe, and a signaling element configured to communicate with the reader and configured to emit a signal destined for a user of the system according to the communication between the reader and the target probe identification element.

### CROSS-REFERENCE TO RELATED APPLICATIONS

This application is the U.S. national phase of International Application No. PCT/EP2022/052309 filed Feb. 1, 2022, which designated the U.S. and claims priority to FR2101522 filed Feb. 17, 2021, the entire contents of each of which are hereby incorporated by reference.

### TECHNICAL FIELD

The present disclosure relates to systems and methods for identifying a target probe from a plurality of probes collaborating with an examination device. Such an examination device may be a medical device, for example an ultrasound wave imaging device. More particularly, the plurality of probes may be ultrasound probes connected or associated with an ultrasound wave imaging device.

### PRIOR ART

An examination device, such as an ultrasound wave imaging device, is typically an imaging device which is intended to provide images of a medium to be examined. In the case of medical applications, this medium is a body, for example a body part of patient (breast, liver, abdomen, . . . ).

A probe is used to observe the medium and generate images of it. The probe is connected to the examination device via a wired or wireless connection for transmitting examination data (for example received wave data or imaging data). The device comprises a processor for processing the examination data, for example for displaying it to a user. The probe and the examination device may form an examination platform. This means that the examination platform may comprise the examination device and one or more probes.

In the case of ultrasound wave imaging, the ultrasonic probe is placed against the outer surface of the medium under observation. In other cases, the probe may be placed close to or inside the observed medium during procedures for example. When the probe is not in use, it is typically placed in a probe-holder of the device.

The device is usually equipped with a plurality of identical or different probes, for different applications. Thus, a plurality of probe-holders are provided. However, the probe-holders often have a unified shape to enable the probes to be supported, whatever their type. As a result, there is often no pre-established order for storing probes in the probe-holders.

These examination (imaging) platforms are often used in dark rooms to make it easier to analyse the images produced. For this reason, the users of the devices may have difficulty finding the instruments they are looking for.

For example, it may be too dark to spot which probe is the one the user needs (i.e. the target probe). In this situation, it may also be difficult to locate an available space for placing a probe, i.e. a probe-holder. It may also be difficult to find the connector between the device and the probe cable, for example for checking the connection.

However, the same problems may occur in normal lighting conditions.

In general it is desirable to carry out examinations efficiently without wasting time, in particular for the well-being of the patient and also to optimise the cost of the examination.

It is known to detect movements of probes held by a support. For example, patent application EP3175794A1 describes an ultrasound diagnostic device comprising a support used for holding a probe; an object sensor for detecting physical changes around the support; an electromagnetic wave sensor for detecting electromagnetic waves around the support; and a controller for determining a port name for a probe held in the support, based on detection values from the object sensor and the electromagnetic wave sensor.

This device comprises a complex sensor system and cannot detect a probe which remains passive, for example does not move or is not activated by the device.

### PRESENTATION OF THE PRESENT DISCLOSURE

The aim of the present disclosure is to identify a target probe of a plurality of probes collaborating with an examination device in a simple and reliable manner. The present disclosure makes it possible to signal the target probe to a potential user. In particular, the aim of the present disclosure is to provide a system which makes it possible to easily equip an examination platform (comprising an examination device and at least one probe) with this functionality for identifying a target probe. In addition, the objective of the disclosed system is in particular to identify and/or locate the target probe which is placed in one of the various probe-holders and to inform the user.

For this purpose, the present disclosure proposes a system for identifying a target probe of a plurality of probes collaborating with an examination device, the system comprising:
an identification element configured to be associated with the target probe, and at least one probe-holder configured to be associated with the examination device and to support the target probe,
a reader associated with the probe-holder and configured to communicate with the identification element of the target probe, and
a signalling element configured to communicate with the reader and configured to emit a signal to a user of the system based on the communication between the reader and the identification element of the target probe.

On this basis, the existing examination platform can be equipped with the system for enabling the identification of a target probe. For example it is sufficient to equip a conventional probe with the identification element, so that it can be located as a target probe in one of the various probe-holders.

The reader may be configured to determine the location of the target probe, for example which probe-holder it is placed in. More generally, the system may be configured to locate the target probe.

The identification element may be configured to send information identifying the target probe to the reader.

The signalling element may be configured to emit a signal as a function of the identification information.

As a result, the signal may include information which represents the identification of the target probe and/or the location of the identified target probe.

The target probe and the examination device may form an examination platform. This means that the examination platform may comprise the examination device and one or more probes comprising the target probe.

The user of the system may also be a user of the target probe and/or the examination device and/or the platform.

The reader may be configured to communicate with a processor of the examination device.

The signalling element may also emit the signal as a function of a control signal received by the processor.

For example, this control signal may comprise a request for a target probe to be identified by the system. Therefore, the control signal may comprise a request for identification and/or highlighting of the target probe.

If the reader communicates with the identification element of the target probe, the signalling element may emit a signal indicating the presence of said probe. This information on presence may also include the location and/or the status of the target probe.

The system may also comprise a plurality of identification elements, each configured to be associated with a probe.

The system may also comprise a plurality of probe-holders.

Each probe-holder may comprise a reader and/or a signalling element. Alternatively, the probe-holders may comprise a centralised reader and/or a centralised signalling element.

The examination device may be equipped for example with a processor and/or a screen. The processor may be connected to one or more probes.

According to one aspect, the central device may be equipped with the probe-holder(s) and/or the reader(s). In one embodiment, each probe-holder comprises a dedicated reader. In this case, it is sufficient to equip the central device with these probe-holders to benefit from the advantages of the present disclosure.

Alternatively, the reader and/or the probe-holder may also be positioned remotely from the device, or the reader may be positioned remotely from or outside the probe-holder. It may be desirable for the reader to be positioned such that the probe-holder is within its reading range.

The signalling element may comprise at least one light source (for example one or more (O)LEDs) and/or sound source (for example a speaker).

If the system comprises a plurality of probe-holders, each probe-holder many comprise a signalling element in the form of at least one light source.

As a result, it is easily possible to signal to the user the location of the target probe, for example by switching on the light source of the probe-holder which supports the target probe.

The signal emitted by the signalling element may comprise an electronic signal configured to present information on a display system and/or to emit sound through a loudspeaker.

The signalling element may comprise at least one interface for transmitting the signal to an external device such as a screen, a tablet and/or a sound device.

The signal emitted by the signalling element may be adapted to warn the user directly, or it may be adapted to be processed by an external electronic device and take the form of an electronic signal. In addition, this external electronic element may be configured to generate a signal for the user based on the electronic signal.

The probe-holder may comprise the reader and/or the signalling element. A probe-holder of this type may be considered to be an “intelligent probe-holder”. As a result, it is sufficient to equip a conventional device with one or more intelligent probe-holders.

The probe-holder may comprise a receptacle configured to receive a probe and/or at least one light source configured to illuminate the probe-holder and/or the receptacle. This light source is an embodiment of a signalling element. It has the advantage that each probe-holder can be equipped with such a light source. In this case, it becomes possible to use these light sources to indicate the location of the target probe.

The probe-holder may comprise at least one additional light source located on the lower side of the probe-holder and/or configured to provide lighting underneath the probe-holder.

This additional light source may be configured to signal additional information to the user.

It can also be configured to illuminate the region underneath the probe-holder, for example the interface connecting the probe to the device.

For example, the probe may be configured to be connected to the examination device by wire or wirelessly.

If the target probe indicated in an identification request is located in a probe-holder but is not connected to the examination device, the additional light source may be illuminated. Therefore, the additional light source can signal to the user that the probe is not connected and/or can be used to connect the probe more easily.

The communication between the reader and the identification element of the probe may be wireless. For example, the communication may comprise at least one of the following technologies: RFID®, NFC®, BLE®, Wifi® or optical communication.

In particular, the reader can be an RFID reader and the identification element can be a passive RFID tag. In this case, as with other alternatives, the identification element may be a passive element without its own power source such as a battery. On the other hand, the identification element may be powered by the signal transmitted by the reader.

Furthermore, the communication between the reader and the identification element of the probe may be performed by an interface other than an interface between the examination device and the probe used to transmit examination data from the probe to the device. Therefore, the system may provide an additional connection between the probe-holder (and therefore for example the examination device) and the probe. This concept has the advantage that a conventional examination device can easily be equipped with the system according to the present disclosure, without any modification of the existing connection and/or interface and/or communication used to transmit examination data from the probe to the device.

The probe-holder (i.e. at least one probe-holder or each probe-holder) may comprise a protective element configured for the electromagnetic protection of the reader. In particular, the protective element can be configured to protect the reader from electromagnetic effects generated outside the receptacle. Thus, the protective element can be configured to prevent the reader from reading an identification element from a probe (i.e. receiving electromagnetic waves from the identification element), which is outside the receptacle and/or which is positioned in another probe-holder or outside the probe-holders.

The protective element can be and/or can comprise at least one electromagnetic insulation wall. The insulation wall can be made for example from a metallic material (or other material capable of blocking electromagnetic waves). The insulation wall can therefore be a metal fold or layer arranged on or in the probe-holder.

The electromagnetic insulation wall can be arranged on at least one lateral side of a shell of the probe-holder. The electromagnetic insulation wall can be arranged on the outside and/or inside of the shell or may form a part or a wall of the shell.

As a result, in the case of a plurality of probe-holders arranged adjacent to one another, it may be sufficient that each probe-holder has an insulation wall on (only) one side. An insulation wall can thus be arranged so as to separate the readers of two adjacent probe-holders. As a result, neither of these two readers can accidentally read an identification element from a probe arranged in the other probe-holder.

It is also possible for the electromagnetic insulation wall to be arranged on a surface of the receptacle.

As a result, the insulation wall can be arranged on at least one (or only one) side wall of the inner side of the receptacle. Alternatively, the insulation wall may be arranged over the entire surface of the receptacle. In this latter case, insulation wall may include an opening where the reader is arranged to enable to the reader to read an identification element of a probe arranged in the receptacle. Alternatively, the insulation wall can enclose the reader such that the reader is capable of reading an identification element of a probe arranged in the receptacle.

In addition, the present disclosure proposes a system for locating a target probe, comprising the system described above, wherein the reader is configured to locate the target probe. For example, the reader may comprise a plurality of reader units in order to locate the position of the target probe.

The present disclosure also relates to an examination device configured to collaborate with a plurality of probes, comprising a processor configured to process examination data received from a probe via an operating connection (for example a data exchange channel between the probe and the probe-holder), and a system as presented above. The examination device can be equipped with at least one probe-holder as set out above.

The operating connection can comprise a wired or wireless connection, for example one or more electrical wires or a wireless connection, for example of the WIFI® type.

The reader can be configured to communicate with the identification element via a different connection than the operating connection.

In particular, if the operating connection can be in the form of wired connection, the other connection can be a wireless connection, for example an RFID connection.

The use of another connection also makes it possible to signal the identification and/or other information from the target probe to the reader (and therefore to the destination of the examination device), even if the probe is not connected to the examination device via the operating connection or if this operating connection is not functioning correctly.

The present disclosure also relates to an examination platform comprising the device as presented above, and at least one probe equipped with an identification element.

The present disclosure also relates to a method for identifying a target probe of a plurality of probes collaborating with an examination device, comprising the following steps:
a communication step in which a reader associated with a probe-holder for supporting the target probe communicates with an identification element associated with the target probe, and
a signalling step in which a signalling element emits a signal to a user (for example from the probe and/or the examination device) as a function of the communication between the reader and the identification element.

The signal can be adapted to indicate to the user the status of the target probe, comprising at least one of the following signal types:
a signal type which indicates in which probe-holder of a plurality of probe-holders of the device the target probe is placed,
a signal type for indicating that the probe is suitable for a predetermined examination or a given phase of an examination,
a signal type for indicating that the target probe is undergoing maintenance,
a signal type for indicating that the probe is out of order,
a signal type for indicating that the target probe is inserted incorrectly into the probe-holder,
a signal type for indicating that the target probe is not connected to the examination device, and
a signal type for indicating that the target probe is in test mode.

These types of signalling to the user can be different, for example by using lights with different colours and/or flashing modes, so that the user can more easily recognise the current status of the target probe.

Alternatively, at least some of the types may be similar.

Prior to the use of the target probe in a predetermined examination, the signalling element can emit a first signal type which indicates in which probe-holder the target probe is placed at a first signalling level.

During use and/or at the end of use, the signalling element can emit a second signal type which indicates which probe-holder(s) is/are suitable for receiving the target probe after use.

The first and second signalling levels can be different, for example by using lights with different colours and/or flashing modes. In another example, the first level can be such that the user's attention is attracted, for example by using a stronger light that makes it possible to find the target probe more rapidly and easily. The first signal type can also comprise a display on a screen of the examination device and/or voice information. The second level can be such that the user's attention is attracted to a lesser degree, for example by using a less bright light or by using a darker colour, in order to prevent the user from being disturbed by the light during the examination. Alternatively, the two levels may be similar.

The method can also comprise the following steps:
equipping each probe with an identification element,
equipping the examination device with probe-holders each comprising a reader and/or a signalling element, or each comprising a central reader and/or a central signalling element, and/or equipping existing probe-holders of the device with readers or a central reader and/or signalling elements or a central signalling element,
recording in a data memory which probe-holder is suitable for receiving which category of probe, and
optionally signalling to the user via the signalling element, if a probe is placed in a probe-holder which is not suitable for this probe.

A system memory or remotely located memory can therefore record probe identifications, which makes it possible to establish a precise identification request for finding a target probe.

In addition, the memory can include mapping of the compatible probe-holders and probe types. It is also possible that the mapping or another device records a current location of the probes in the probe-holders. However, this mapping is not obligatory, as the present disclosure makes it possible to locate each probe (target) at any moment. Updating the current location in a memory is therefore not necessary but is still possible.

Furthermore, the present disclosure makes it possible to signal to the user when a probe is placed in a probe-holder not suitable for this probe, even if the probe is not activated and/or not connected to the examination device by the operating connection.

The present disclosure also relates to an examination method, comprising,
in addition to the steps set out above,
acquiring examination data via the target probe which is a first target probe,
determining via a processor of the device the recommendation for acquiring additional examination data based on the examination data acquired by the first target probe,
identifying at least one second target probe suitable for acquiring additional examination data and
signalling to the user in which probe-holder the second probe is placed.

The present disclosure can therefore use an algorithm which automatically determines whether the data acquired by the (first) target probe is sufficient for the examination or whether additional examination data would be useful, for example to improve the precision of the examination or to handle a part of the examination not yet covered (for example a change in the imaged organ). In this case, the algorithm can also determine a second target probe suitable for acquiring additional examination data.

Finally, the algorithm can use the system according to the disclosure in order to signal to the user the current location of this second target probe.

This algorithm can comprise or can be based on an artificial intelligence algorithm, for example an intelligent learning algorithm and/or at least one neural network.

An intelligent learning algorithm can be used for determining the recommendation for acquiring additional examination data. This intelligent learning algorithm can use the examination information as input data and determine as output data an identification of an possible second target probe and/or an identification of the probe-holder comprising the second target probe.

For example, this algorithm can comprise two parts:
a first part can comprise for example at least one convolutional neural network which estimates (for example by regression of examination data acquired by the first target probe) at least one quality parameter of examination data and/or a completeness parameter of the examination (for example when two probes are needed for imaging two different organs during the same examination), and
a second part can comprise a neural network which is configured to classify the probes based on the (at least one) examination data quality parameter, in order to identify the second target probe and optionally identify the probe-holder comprising the second target probe.

The features and advantages of the present disclosure will become apparent from the following description, given solely by way of a non-limiting example, and made with reference to the accompanying figures. In particular, the examples illustrated in the figures may be combined.

### BRIEF DESCRIPTION OF THE FIGURES

FIG. 1 is a schematic view of a system for identifying a target probe according to an embodiment of the present disclosure.

FIG. 2a shows schematically a side view of a first example of a probe-holder according to the present disclosure, in particular comprising a reader and a signalling element.

FIG. 2b shows schematically a side view of a second example of a probe-holder according to the present disclosure, in particular equipped with a wireless connection for communicating with the examination device.

FIG. 2c shows schematically a perspective view of an example of a probe-holder according to the present disclosure, in particular of the first or second example.

FIG. 2d shows schematically a perspective view of an example of a probe-holder with a first example of a protective element according to the present disclosure, in particular on a lateral side of a shell of the probe-holder.

FIG. 2e shows schematically a perspective view of an example of a probe-holder with a modification of the first example of a protective element according to the present disclosure.

FIG. 2f shows schematically a perspective view of a second example of a protective element according to the present disclosure, in particular consisting of the shell of the probe-holder.

FIG. 2g shows schematically a perspective view of an example of a probe-holder with a third example of an protective element according to the present disclosure, in particular in the receptacle of the probe-holder.

FIG. 2h shows schematically a perspective view of an example of a probe-holder with a modification of the third example of an protective element according to the present disclosure.

FIG. 2i shows schematically a perspective view of an example of an probe-holder with a fourth example of an protective element according to the present disclosure, in particular on an outer surface of the receptacle of the probe-holder. FIG. 3 shows schematically an example of a probe equipped with an identification element according to the present disclosure, in particular with an RFID tag.

FIG. 4a, FIG. 4b, FIG. 4c, and FIG. 4d show schematically elevated views of different examples of an examination device according to the present disclosure.

FIG. 5 shows schematically an example of a method according to the present disclosure, in particular an identification of a target probe and signalling for the attention of the user.

### DESCRIPTION OF EMBODIMENTS

In the various figures, provided by way of illustration, the same reference numerals denote identical or similar elements.

FIG. 1 is a schematic view of a system 10 for identifying a target probe according to one embodiment of the disclosure. The system comprises an identification element 1 configured to be associated with the target probe (not shown in FIG. 1).

In addition, the system comprises at least one probe-holder 2 configured to be associated with the examination device (not shown in FIG. 1) and to support a target probe. The probe can be attached to, deposited in, placed on or received by the probe-holder when not in use. On the other hand, when it is to be used, for example during an examination or for a test/treatment of the probe, it can be removed from the probe-holder. The probe-holder can mainly be considered to be a stationary element and/or the probe as a mobile element. The probe can therefore be moved relative to the probe-holder (see continuous arrow in the figure).

In addition, the system comprises a reader 3 associated with the probe-holder 2 and is configured to communicate with the identification element of the target probe 1 (cf. flash with dotted line). Furthermore, the system 10 comprises a signalling element 4 configured to communicate with the reader 3 and configured to emit a signal to a user of the system as a function of the communication between the reader 3 and the identification element 1 of the target probe.

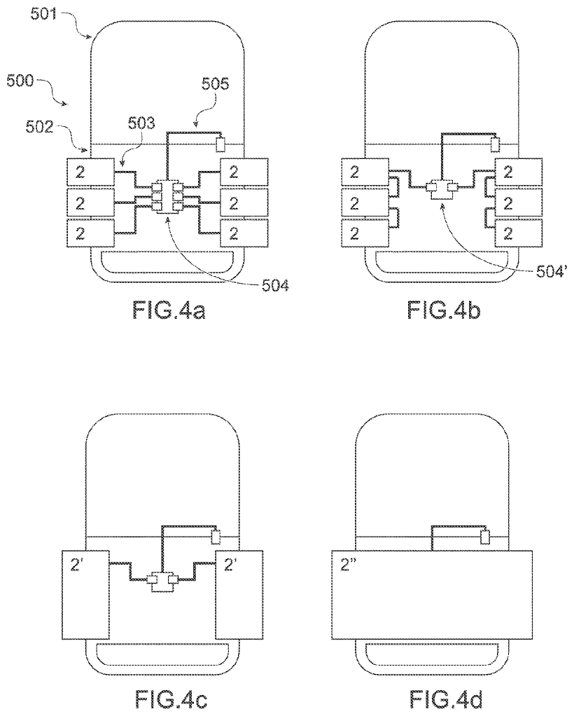

The probe-holder 2 can comprise the reader 4 and/or the signalling element 3. This type of probe-holder can be considered to be an “intelligent probe-holder”, as described in more detail with reference to FIGS. 3a-3c, and 4a-4d. Alternatively, the reader 4 and/or the signalling element 3 can be elements external to the probe-holder 2. For example, the reader can be located only in the proximity of the probe-holder, such that it can read the identification element of a probe located on the probe-holder or of a plurality of probes located on different probe-holders. The signalling element 4 can also be connected by wire or wirelessly to the reader 4. The signalling element 4 can also be part of the reader 3.

More generally, the system of the present disclosure makes it possible to equip an existing examination platform (i.e. a conventional platform comprising an examination device and one or more probes) with the functionality of identifying a target probe according to the present disclosure. It is sufficient to equip a conventional probe with the identification element 1 and an examination device with the probe-holder 2 which comprises or is associated with the reader 3 and/or the identification element 4.

It is also possible to provide a system 10 with a plurality of identification elements 1 (for example one per probe) and a plurality of probe-holders 2 (for example one per probe or a predefined number). In this case, each probe of the examination device can be identified and therefore located in one of the probe-holders 2.

The reader can be configured to locate a target probe in one of the probe-holders 2. For example, each probe-holder can be equipped with its own reader. In this case, the reader that can read and therefore identify the target probe can also locate it in its probe-holder. In one variant, a reader system can be provided which can locate a target probe in an area comprising a plurality of probe-holders.

The reader 3 can also be configured to communicate with a processor of the examination device, for example by wired or wireless connection. It is therefore possible that this processor sends a control signal comprising a request for a target probe to be identified by the system. The processor may indicate to the system 10 (or the reader 4) which target probe is to be identified and/or located.

If the reader communicates with the identification element of the target probe, it can locate it in one of the probe-holders. The signalling element 4 can emit a signal indicating the presence of said probe. This presence information can also include the location of the target probe.

The signal emitted by the signalling element may comprise light and/or sound. If the system comprises a plurality of probe-holders 2, each probe-holder can comprise a signalling element 4 in the form of at least one light source. As a result, it is easily possible to signal the location of the target probe to the user, for example by switching on the light source of the probe-holder 2 supporting the target probe.

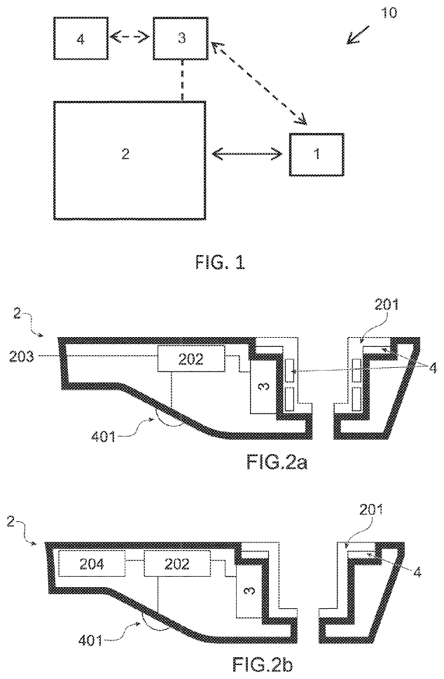

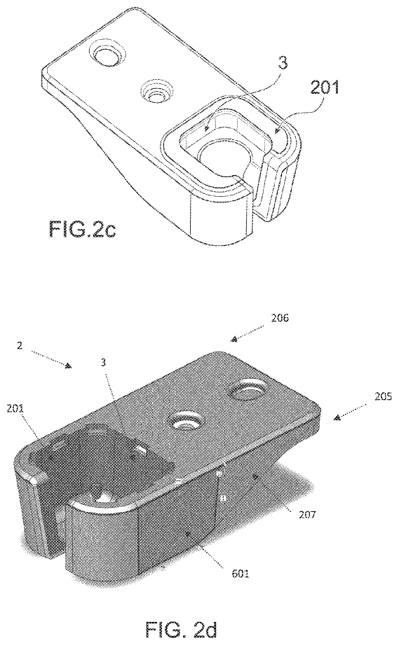

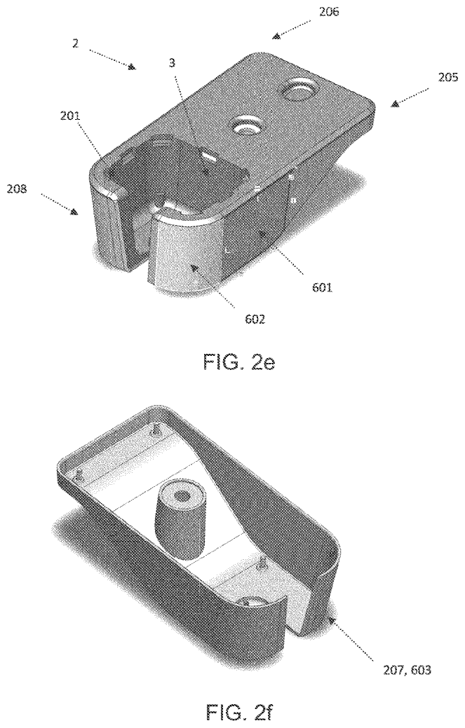

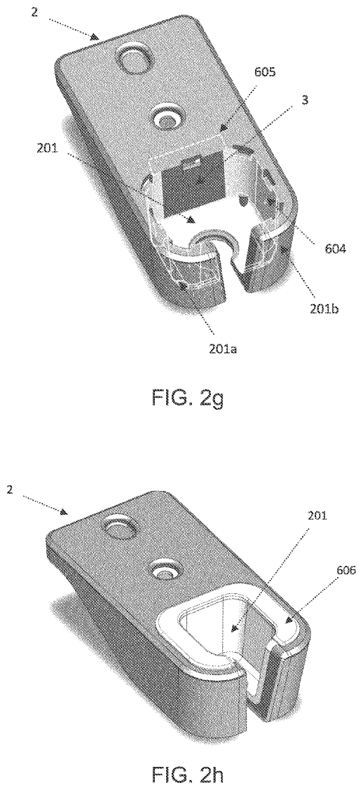

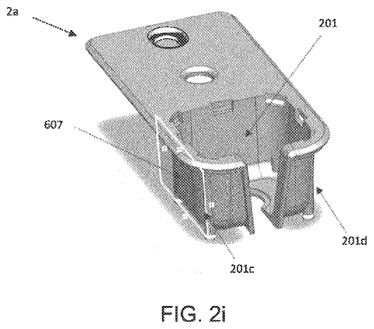

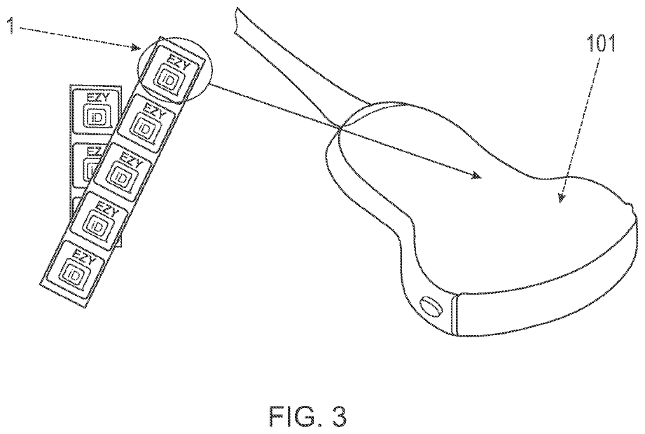

FIG. 2a shows schematically a side view of a first example of a probe-holder according to the present disclosure, in particular comprising a reader and a signalling element. In particular, FIG. 2a shows a first example of an “intelligent” probe-holder 2 comprising a reader 3 (for example an RFID antenna) and a signalling element 4 (for example one or more LEDs, in particular multi-coloured LEDs). The LEDs are placed close to and/or around a receptacle 201 which is configured to contain/support/maintain a probe. The receptacle can be partially or totally translucent or even transparent.

The probe-holder can also comprise an electronic circuit 202 (for example a printed circuit board PCB) configured to communicate via a connection 203 with an examination device (not shown in FIG. 2a). The electronic circuit 202 can also be configured to control and power the signalling elements 4, 401 and/or the reader 3. For example, a processor of the examination device may send a command (i.e. an identification request) to the reader 3 via the circuit 202 in order to find a target probe. The reader can send a command to the signalling elements 4 and/or 401 via the circuit 202 in order to transmit a signal to a user.

The intelligent probe-holder can therefore be configured to identify the probe which is placed in its receptacle and to visually inform the user of information on this probe if necessary. To achieve this, the probe-holder can use RFID technology for example. It can contain an RFID reader 3 equipped with an antenna (optionally the RFID reader 3 can be used to update the tag 1) in its internal volume and so as to be able to pick up an RFID tag located in the volume of the receptacle or on its perimeter. More specifically, the RFID technology selected could be NFC (near-field communication) technology. The NFC tag 1 can operate passively or actively. The passive operation has the advantage of being easier to use, in particular in terms of connectivity and reduced weight (very important advantage for the ergonomics of ultrasound probes).

In the most developed version, a light source 401 can be provided located underneath the probe-holder directed towards the probe connectors to allow better visibility of the connectors when it is necessary to connect or disconnect a probe. For example, this light source would not be active during examinations (i.e. when a probe is activated and when in use) so as not to create light pollution. In one variant, the light source of the connectors can be adjusted either manually or automatically.

In the example of FIG. 2a, it is the receptacle itself which can be luminous or illuminated. Indeed, by making a translucent receptacle 201 the incorporation of LEDs 4 into the interior allows it to be backlit entirely (or partly). The semi-transparent material of the receptacle can be a hard plastic, such as for example ABS, or can be a more flexible material such as silicone. This variant can also be made using black light (UV) LEDs which, coupled with a fluorescent and semi-transparent material, would advantageously provide low-glare lighting for the user.

The behaviour can be customised by the user (e.g.: choice of LED colour, choice of flash duration, type of continuous/discontinuous lighting, LED intensity, etc.). Predefined lighting effects can also be selected by the user. For example, the user can choose a moving light effect by switching on/off several probe-holders one after the other to give the illusion of a path.

The FIG. 2b shows schematically a side view of a second example of a probe-holder according to the present disclosure, in particular equipped with a wireless connection for communicating with the examination device. In broad terms, the example of FIG. 2b corresponds to the first example of FIG. 2a. However, in FIG. 2b the probe-holder can have its own power source so as to be autonomous. For example, it may comprise one or more batteries 204.

In addition, the electronic circuit can comprise a wireless interface (for example an antenna, for example using Bluetooth or similar technology) to communicate with the examination device. Thus, the probe-holder only needs to be mechanically connected to the examination device or to another stationary element or virtually stationary element (if the examination device is located at a distance from the probe(s)) for use.

Furthermore, FIG. 2b shows another example of positioning of LEDs 4 below the receptacle 201, in particular with LEDs located around and below the receptacle. In one variant the LED(s) can be located above the receptacle 201. The light from these LEDs is therefore visible to the user. In another variant, one or more LEDs 4 can be arranged on the plastic shells of the probe-holder. In a developed version of the present disclosure, the visual information is for example LED backlighting (other less relevant technologies are also possible, such as small incandescent bulbs) which can vary in colour.

FIG. 2c shows schematically a perspective view of an example of a probe-holder according to the present disclosure, in particular the first or second example. This example shows the positioning of the reader 3 in the proximity of the receptacle 201, the reader 3 being inside the probe-holder it is not visible in this view. The reader 3 can therefore be close to a probe located in the receptacle, in particular close to the identification element of the probe. This enables a reliable communication between the reader the identification element.

FIG. 2d shows schematically a perspective view of an example of a probe-holder 2 with a first example of a protective element 601 according to the present disclosure, in particular on a lateral side 205, 206 of a shell of the probe-holder.

In general, the probe-holder 2 can therefore comprise a protective element 601 configured for the electromagnetic protection of the reader. In particular, the protective element can be configured to protect the reader from electromagnetic influences generated outside the receptacle. As a result, the protective element can be configured to prevent the reader from reading the identification element of a probe outside the receptacle and/or which is positioned in another probe-holder or outside the probe-holders. This means that the protective element can be configured to prevent the reader from receiving electromagnetic waves from this identification element.

The protective element can be and/or can comprise at least one electromagnetic insulation wall. The insulation wall (i.e. the protective element) can be made for example from a metallic material (or other material capable of blocking electromagnetic waves). The insulation wall can therefore be a metal fold or layer arranged on or in the probe-holder.

Other examples of materials and methods of producing a protective element comprise:
a fold of aluminium (or made from any other material capable of blocking electromagnetic waves), in particular adapted to the shape of the probe-holder. The fold can also be referred to as a screening fabric;
a grid made from a material capable of blocking electromagnetic waves, in particular with a grid size adapted to the wavelength of the electromagnetic waves emitted by the identification elements;
coating part of the probe-holder (for example the shell or parts thereof, e.g. made of plastic material) with a metallic layer.

The electromagnetic insulation wall can be arranged on at least one lateral side 205 and/or 206 of a shell 207 of the probe-holder 2. The electromagnetic insulation wall can be arranged outside and/or inside the shell 207 or can form a part or wall of the shell 207.

As a result, in the case of a plurality of probe-holders arranged adjacent to one another (cf. for example FIG. 4a, 4b), it may be sufficient for each probe-holder to have an insulation wall on (only) one side 205 or 206. However, each pair of adjacent probe-holders can be separated by an insulation wall. As a result, neither of these two readers can accidentally read an identification element from a probe arranged in the other probe-holder.

FIG. 2e shows schematically a perspective view of an example of a probe-holder 2 with a modification of the first example of a protective element according to the present disclosure. In addition to the electromagnetic insulation wall 601, the protective element may comprise an additional electromagnetic insulation wall 602 on a front side 208 of the probe-holder 2. The additional electromagnetic insulation wall 602 can also be an extension of the (first) insulation wall 601, i.e. be the same wall. The additional electromagnetic insulation wall 602 can also be arranged on one or on two sides 205, 206 of the probe-holder.

Thus the electromagnetic protection of the reader 3 can be improved, in particular with regard to undesirable electromagnetic waves which originate rather from a front side than a lateral side of the probe-holder 2.

FIG. 2f shows schematically a perspective view of a second example of a protective element 603 according to the present disclosure, in particular the shell 207 of the probe-holder. As a result, the complete shell 207 can comprise an electromagnetic insulation wall 603, for example in the form of a layer. It is also possible for the shell to be made from the electromagnetic insulation material.

FIG. 2g shows schematically a perspective view of an example of a probe-holder with a third example of a protective element 604 according to the present disclosure, in particular in the receptacle 201 of the probe-holder 2. It is thus possible for the electromagnetic insulation wall 604 to be arranged on a surface of the receptacle 201.

As a result, the insulation wall 604 can be arranged on at least one (or only one) side wall 201a, 201b of the inner side of the receptacle 201. Alternatively, the insulation wall can be arranged over the whole surface of the receptacle. In this latter case, the insulation wall can include an opening where the reader is arranged to enable the reader 3 to read an identification element of a probe arranged in the receptacle. Alternatively, the insulation wall can enclose the reader such that the reader is capable of reading an identification element of a probe arranged in the receptacle (cf. the extension 605 of the wall).

FIG. 2h shows schematically a perspective view of an example of a probe-holder with a modification of the third example of a protective element according to the present disclosure.

The receptacle 201 can comprise on top of the insulation wall 604 a further layer 606, for example made of rubber or another flexible material. Said layer may be configured to come into contact with a probe placed in the receptacle of the probe-holder. The layer can therefore be configured to protect the probe from scratching and/or to ensure that the probe is held reliably in the probe-holder 2.

FIG. 2i shows schematically a perspective view of an example of a probe-holder with a fourth example of a protective element 607 according to the present disclosure, for example on an exterior surface 201c, 201d of the receptacle 201 of the probe-holder. As a result, an electromagnetic insulation wall 607 can be arranged inside the probe-holder 2, for example on the inner surface of the outer shell (not shown in FIG. 2i) or on the outer surface of the inner shell 2a providing the receptacle 201 or anywhere between the two surfaces. In addition, the electromagnetic insulation wall 607 can be arranged on one or two sides 201c and 201d.

FIG. 3 shows schematically an example of a probe 101 equipped with an identification element according to the present disclosure, in particular an RFID tag.

The probe 101 may be a conventional probe, for example a probe known from the prior art. In this case the probe may be equipped with an identification element 101, for example an NFC tag in the form of a sticker placed on the inner or outer face of the probe shells. A conventional probe can therefore only be modified by the attachment or integration of an identification element (for example a passive RFID tag).

Each probe can therefore be equipped with its own identification element which enables unique identification of this probe. Each identification element can identify for example the serial number of the probe (referred to as the probe ID) and/or its probe type and possibly contextual information such as:
the hospital establishment to which it belongs, and/or identification of the examination device to which the probe is connected. Ideally, these tags can be passive and they do not need any energy of their own to operate (apart from that induced by the RFID/NFC 3 reader when they are “read”).

In one variant, the identification elements can also contain a rewritable NFC chip so that information can be rewritten if necessary (for example, change of establishment after reconditioning, last tests carried out and their results, coded history of use). The information can be rewritten wirelessly using the magnetic field emitted by the rewriting module (confused with the reader 3). This rewriting can also enable the writing of data from inner sensors of the probe such as for example a temperature sensor, a shock sensor or any other sensor that might be useful to incorporate in a probe.

The reader 3 (for example an NFC antenna) of the probe-holder can detect the identification element (the NFC tag) of the probe when it is placed in the receptacle. The information retransmitted to the device, which by means of its software sends a command to the visual information device, such as for example an LED. The information can be highlighted in other ways, such as with a sound signal (location sound or recorded or synthetic voice indicating which probe-holder the probe is in) or a visual signal displayed on the screen.

In some variants, the communication technologies from the probe to the probe-holder may be different than RFID, such as for example Bluetooth, BLE, Wifi or even optical communications (in this specific case, the communication antenna in the probe-holder is replaced by an optical reader capable of reading a barcode or a QR code located on the outer surface of the probe shell and the code is read when the probe is positioned in the receptacle).

FIGS. 4a to 4d show schematically plan views of different examples of an examination device 500 according to the present disclosure. In each example, the examination device 500 can comprise a central device 501 (comprising for example a processor for processing examination data) and a part 502 used primarily as a user interface (i.e. as a control panel, comprising for example one or more screens, for example tactile or non-tactile screens, and a keypad or other control elements). This part 502 can also comprise one or more probe-holders 2, which facilitates the accessibility of probes stored in probe-holders for a user of the device 500.

The device can therefore comprise a plurality of probe-holders 2 according to the disclosure. These probe-holders can be added to a conventional examination device, for example by replacing conventional probe-holders of the device.

Optionally, the examination device can be equipped with a microphone to enable the use of voice commands. This microphone can be added as an additional module onto an existing examination device, connected to a USB port for example.

In the first example illustrated in FIG. 4a the examination device 500 comprises a plurality of probe-holders 2 according to the disclosure. A probe-holder 2 (in particular each probe-holder 2) can have a power supply and a communication channel to the examination device. In a mode of operation according to one example, the communication and the power supply are provided via a USB cable 503 connected to the control panel 502 of the device 500. A USB multisocket 504 can then be positioned and fixed underneath the control panel 502, with 6 to 8 female USB ports for connecting the probe-holders 2 for example. The USB multisocket 504 can be connected to a USB port 505 of the examination device and therefore to a processor of the device for example.

According to one variant of the connection according to an example illustrated in FIG. 4b, a single USB multisocket 504′ equipped with one or two ports is suitable for connecting the probe-holders 2 to it, the latter being connectable via a removable cable to the probe-holders 2 juxtaposed thereto.

In a variant shown in FIGS. 4c and 4d, the probe-holders can be merged to form multiple modules 2′ and 2″. For example, a triple probe-holder 2′ can be the fusion of three single probe-holders 2 and would therefore in this illustrative example contain three receptacles positioned on the same side of the control panel (see FIG. 4c). In another example, the multiple probe-holder 2″ is the fusion of six single probe-holders 2 whose receptacles are positioned on either side of the control panel (see FIG. 4d).

In another embodiment, the communication between a probe-holder 2″′ and the examination device can be achieved via a wireless connection, for example via a Bluetooth connection. A suitable antenna can therefore be installed in the probe-holder 2″′, for example together with a power source. Therefore, the probe-holders can be powered by cells or batteries for example. Thus, the probe-holder is fully wireless and easier to install when installing on a conventional examination device.

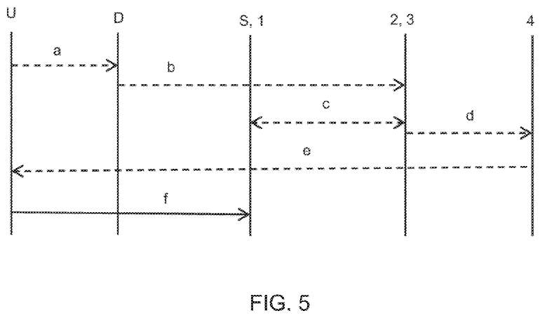

FIG. 5 shows schematically an example of an method according to the present disclosure, in particular the identification of a target probe and signalling to the user.

In step “a” the user starts or prepares an examination procedure with an examination device D. For example, the user may select and activate a target probe S (for example on a user interface of the examination device D). Alternatively, a processor of the examination device may automatically select a target probe S (for example according to a predefined algorithm and/or an algorithm using artificial intelligence) based on a gesture/command/action of the user and/or the context of a procedure in progress.

In step “b” the examination device D sends a request to the reader 3 and/or the probe-holder 2 concerning a target probe S to be identified and/or located. This request can be a message comprising the identification of the target probe S. If there are several probe-holders, each equipped with a reader, the examination device D can also send the request to each reader 3.

In step “c” the reader(s) 3 (or the probe-holder(s) 2 comprising the reader(s) 3) communicate with the identification element(s) 1 of the probes comprising the target probe S. For example, the reader(s) 3 can send a signal to each probe requesting their identification. In response, the probe(s) transmit their identification. The reader(s) can then determine, based on these responses, which probe corresponds to the target probe by comparing the request from the examination device D with the identifications received.

In step “d” the reader(s) 3 (or the probe-holder(s) 2 comprising the reader(s) 3) inform the signalling element 4 of the result of the communication with the identification element(s) 1 of the probes comprising the target probe S. In one example, where each probe-holder comprises a reader, the reader of the probe-holder, which supports the target probe, can communicate to the signalling element 4 the location of the target probe (and for example other readers can thus avoid communication with the signalling element 4).

In response, in step “e”, the signalling element 4 can emit and/or signal to a user information from the target probe resulting from the communication of step “c” (for example the location of the target probe or other target probe status information, as described below). For example, if the signalling element 4 comprises at least one light source on each probe-holder, the light source of the probe-holder supporting the target probe can be activated.

In the scenario where each probe-holder 2 comprises its own reader 3 and its own signalling element 4 (for example one or more (O)LEDs), each probe-holder 2 can perform steps “c” to “e” in an autonomous manner. It is also possible that each probe-holder 2 performs step c, and only this probe-holder 2, which has communicated with the probe-holder, performs steps “d” and “e”.

Lastly, in step “f” the user U can perform an operation with the target probe S as a function of information received by the signalling element 4. In a case according to one example, the user can be informed of the probe-holder currently supporting the target probe S in step “e”. The user can therefore take this probe (for example to carry out an examination or a treatment or a test on this probe). In another example, the user can be informed that the target probe is a probe which is not correctly placed/attached/inserted/connected to the probe-holder, for example by a light source flashing in a predefined colour. In response, the user can connect/attach/place/insert it correctly in step “f”.

However, the information transmitted to the user in step “e” can also be based on determinations of the examination device. For example, the device can determine that the target probe is not correctly connected to the central device. In this case, the user can be informed of the location of the target probe and its status (for example the status of its connection).

However, the present disclosure is not limited to a method for identifying and/or locating a target probe, in order to perform an examination with this target probe.

The present disclosure can also comprise other methods (or modes of use) according to the following examples.

In the following, a mode of use according to one example is presented for locating an activated target probe which is positioned in a probe-holder.

The probe-holder is illuminated by a blue backlight for example to facilitate finding the target probe in a dark room. The probe-holder switches off when the probe is removed from the receptacle by the user. Other probes that may be useful for the examination (for example: of the breast) are temporarily illuminated at a reduced intensity by their probe-holder. The colours may vary as a function of the examination (for example: “the breast” in pink or “the liver” in yellow) and/or the part of the examination in progress (for example: different clinical applications within the same examination).

A mode of use according to an example during the use of the probe according to one example:

The probe-holders with empty receptacles are illuminated slightly in blue or green at the end of the examination or when a probe is changed during the same examination to indicate to the user that they are available for the target probe (the end of the examination being triggered via the user interface or by clicking on the “freeze” button or by an artificial intelligence module assisting with the examination or by the user's wish to change probe signalled via the user interface). As the probe-holders have different receptacle sizes (corresponding to the probes that they can accommodate), only empty receptacles are illuminated that can accommodate the probe currently in use.

A mode of use according to one example in which there is a risk that a probe may fall out:

When a user places a (target) probe into a probe-holder with an unsuitable receptacle, there is a risk that the probe may fall out, often causing breakage. The probe-holder, for which probes compatible with its receptacle are identified, therefore starts flashing to alert the user. The other probe-holders compatible with this probe are illuminated slightly to signal to the user where to place the probe until the change is made.

A mode of use according to one example in which the target probe is not connected:

When a user selects an examination program with a probe type via the user interface, the probe-holder is able to locate the target probe and warns the user that it is not connected by flashing. In one variant, by having a lighting system located underneath the probe-holder, the ultrasound scanner probe connector(s) are illuminated to facilitate the connection and disconnection of the probes. In one variant, this lighting is not active during the examination so as not to create too much light pollution.

A mode of use according to one example during conventional protocols:

The protocols are examination programs of the examination software (for example of ultrasound) guiding the users (by following specific steps according to different examinations). When a protocol is launched, the probe-holders advantageously improve user guidance by indicating the position of the probe to be used, which can vary at each step of the examination, in addition to displaying information on the screen. The user gains in terms of the speed of examination.

A mode of use according to one example involving protocols controlled by artificial intelligence:

These are protocols which are activated according to what is detected by artificial intelligence in order to provide the most suitable examinations for each patient. The automated nature of the launch of these protocols makes highlighting the probe to be used during the examination of even more interest (the user does not need to think).

Example 1: A user examines a patient and identifies an area of interest (for example a lesion). The user can appreciate being guided through the examination in order to increase its relevance, reliability and speed. Artificial intelligence recognises “this case” (for example because it has “learnt” it). On this basis it then recommends a target probe to the user. It uses the present disclosure to illuminate the intelligent probe-holder(s) supporting the target probe.

Example 2: The user sees a small, very superficial lesion with probe A. Artificial intelligence recommends measuring it with a second target probe which is more suitable for superficial lesions. The probe-holder supporting the second target probe is illuminated by means of the present disclosure.

A mode of use according to one example when the probe is undergoing maintenance or at the start-up of the examination device or between each examination:

When a quick test is carried out to check the target probe, the probe-holder may flash, for example with an orange colour, to let the user know that a test is in progress and that the probe cannot be used. When the examination device is undergoing maintenance, either in person by a technician or remotely, the probe-holders can emit a different light than in previous cases, for example orange. When one of the maintenance tests has detected an error on a probe, its probe-holder may light up in red.

A mode of use according to one example during procedures:

During an ultrasound examination, users (for example a doctor) engage in a biopsy or fine needle aspiration (FNA) type procedure. They will ask their assistant to help them manipulate the probes and the ultrasound device. They will use a specific biopsy mode and entrust the handling of the device to the assistant to free up at least one hand for the procedure (for example for holding the needle). The probe-holder accommodating the probe required for the intervention (identified/activated via the biopsy mode or identified via a voice command from the radiologist or via the probe selection screen/buttons or other human-machine interface system) is illuminated, the assistant can quickly hand it to the doctor. The doctor places the needle in the tissue to be sampled at the same time as holding the probe. Once the biopsy has been completed, the doctor hands the probe to the assistant to put back into the probe-holder which lights up according to the last probe used (probe-holder suitable for the type of probe and/or the positioning preferences of the doctor) while proceeding to the extraction of the target tissue contained in the biopsy gun and caring for the patient (for example in a breast biopsy). The doctor and the assistant have advantageously gained examination time and concentration during the procedure, the assistant having thus been guided in the choice of probe by the system or method according to the present disclosure.

In the case of a surgeon in the operating theatre (cardiology, mastectomy, removal of thyroid nodules), it is preferable that the surgeon does not manipulate the probe at all (sterilisation level lower than that of the surgical equipment) so as not to contaminate their hands. The assistants must therefore handle the ultrasound probes themselves. The probe-holders according to the present disclosure therefore facilitate this operating mode by guiding the assistants without involving the surgeon. The surgeon requests the use of probe A by voice command or initiates a specific surgical mode. The probe concerned is activated. The probe-holder containing the probe lights up and an assistant quickly locates it and positions the probe on the patient. The surgeon then proceeds with operations under ultrasound control. If necessary, the surgeon can change probe by voice command or during surgical mode, thus probe A is deactivated and probe B is activated. An empty probe-holder for receiving probe A lights up until the assistant can remove it, then the probe-holder containing probe B lights up. If the probe has not been disinfected as explained below, it will not be offered for use until it has been disinfected and will therefore not be illuminated, even if it is the type of probe requested.

A mode of use according to one example during the preparation and disinfection phase of the ultrasound scanner and probes:

The user (for example a technician) activates the “cleaning” mode or “disinfection” mode. The probes authorised to be disconnected (as they are not undergoing maintenance or testing) are illuminated in green by their respective probe-holder. The probes which are not to be disconnected are illuminated in red by their respective probe-holder. The technician can therefore retrieve and set aside the probes for disinfection, in particular for example in the case of endocavitary probes which have to be placed in a disinfection unit separate from the ultrasound examination device. To replace the probes after cleaning, the second phase of the disinfection mode is activated and the probe-holders light up successively (potentially once the probes are connected to the system) to place the probes in the right place, i.e. in suitable probe-holders. If necessary, a message indicating the procedure to follow for the probe is displayed on the screen in order to guide the user. In one variant in which the probes are wired or wireless, a protocol running in the system or examination device via an artificial intelligence module for example guides the technician in repositioning the cleaned probes. This means that the system or the examination device displays the probe to be positioned on a screen while the corresponding probe-holder is illuminated. The positionings described above are advantageously also linked to user preferences (which can be personalised) and/or to the adaptation of the probe-holders to probes of different types and/or to the positioning of the connectors used or to be used.

A mode of use according to one example during the phase of setting user preferences by the administrator or the user (registration, personalisation or settings):

One of these two persons starts up the personalisation cycle of the examination device, via the desired settings function.

To achieve this, the user selects the settings, either globally (probes to be used preferably placed in the probe-holders located on the left, . . . ) or probe by probe and in this case the system proposes using a probe compatible with the chosen setting as a function or not of the probes currently connected to the examination device: the screen then displays the probe to be positioned and the examination device lights up the compatible probe-holders. The user (individual configuration) or the administrator (collective configuration) can then place the probe in one of these compatible and empty probe-holders and validate the choice via the user interface, then moves to the following probe if necessary. Once the set-up is complete, all of the choices are saved on the user's profile or the collective profile of the institution that owns the device for example. Thus, at the start of a vacation of a given user, a technician or the user can come and position the probes according to said user's preferences. A module of the system or examination device then makes it possible to display successively the probe to be positioned and illuminate the corresponding probe-holder. The technician (and/or the user) thus saves pre-examination preparation time, improves comfort and reproducibility (simplified and accelerated preparation procedure due to lighting). The examination is thus more reliable, improved and optimised.

In general, the user of the system according to the disclosure can be a person who is qualified to perform examinations (for example a doctor or a surgeon) or an unqualified person who can be guided by the system according to the disclosure.

All of these embodiments and other examples such as those described above are given solely by way of non-limiting example and may be combined and/or modified within the scope of the following claims.

1. A system for identifying a target probe (1) of a plurality of probes collaborating with an examination device, the system (10) comprising:
an identification element configured to be associated with the target probe (1), and at least one probe-holder (2) configured to be associated with the examination device and to support the target probe (1),
a reader (3) associated with the probe-holder (2) and configured to communicate with the identification element of the target probe (1),
and a signalling element (4) configured to communicate with the reader (3) and configured to emit a signal to a user of the system (10) as a function of the communication between the reader (3) and the identification element of the target probe (1).

2. The system according to claim 1, wherein the identification element is configured to send identification information of the target probe (1) to the reader (3), and the signalling element (4) is configured emit a signal as a function of the identification information.

3. The system according to claim 1,
wherein the reader (3) is configured to communicate with a processor of the examination device, and
the signalling element(4) emits the signal in addition as a function of a control signal received by the processor.

4. The system according to claim 3, wherein the control signal comprises a request for identification of the target probe (1), and, if the reader (3) communicates with the identification element of the target probe (1), the signalling element(4) emits a signal indicating the presence of said probe.

5. The system according to claim 1,
wherein the system (10) comprises a plurality of identification elements, each configured to be associated with a probe, and/or
the system (10) comprises a plurality of probe-holders, wherein each probe-holder comprises a reader and/or a signalling element, or the probe-holders comprise a centralised reader and/or a centralised signalling element.

6. The system according to claim 1,
wherein the signal emitted by the signalling element (4) comprises light and/or sound, and/or
the signalling element (4) comprises at least one light source (401) and/or sound source, and/or
if the system (10) comprises a plurality of probe-holders, each probe-holder (2) comprises a signalling element in the form of at least one light source.

7. The system according to claim 1,
wherein the signal emitted by the signalling element comprises an electronic signal configured to present information on a display system and/or to emit sound through a loudspeaker, and/or
the signalling element comprises at least one interface for transmitting the signal to an external device.

8. The system according to claim 1,
wherein the probe-holder (2) comprises the reader (3) and/or the signalling element, and/or
the probe-holder (2) comprises a receptacle (201) configured to receive the probe, and/or at least one light source configured to illuminate the probe-holder (2) and/or the receptacle (201).

9. The system according to claim 1, wherein the probe-holder (2) comprises at least one additional light source located on the lower side of the probe-holder (2) and/or configured to provide illumination underneath the probe-holder (2).

10. The system according to claim 1,
wherein the probe is configured to be connected to the examination device by wire or wirelessly, and/or
if the target probe (1) indicated in an identification request is located in a probe-holder (2) but is not connected to the examination device, the additional light source is switched on.

11. The system according to claim 1,
wherein the communication between the reader (3) and the identification element of the probe is wireless and/or comprises at least one the following technologies: RFID, NFC, BLE, Wifi or optical communication, and/or
the reader (3) is an RFID reader and the identification element is a passive RFID tag, and/or
the communication between the reader (3) and the identification element of the probe is performed via an interface different from an interface between the examination device and the probe used to transmit examination data from the probe to the device.

12. The system according to claim 1, wherein
at least one or each probe-holder (2) comprises a protective element configured for the electromagnetic protection of the reader (3), and/or configured to prevent the reader (3) from reading the identification element of a probe which is outside the receptacle (201) and/or which is positioned in another probe-holder (2).

13. The system according to claim 12, wherein the protective element is and/or comprises at least one electromagnetic insulation wall.

14. The system according to claim 13,
wherein the electromagnetic insulation wall is arranged on at least one lateral side of a shell of the probe-holder, and/or
the electromagnetic insulation wall is arranged on a surface of the receptacle.

15. The system according to claim 1, wherein the reader (3) is configured to locate the target probe (1).

16. An examination device configured to collaborate with a plurality of probes, comprising:
a processor configured to process examination data received from a probe via an operating connection, and
the system according to claim 1,
wherein the examination device is equipped with at least one probe-holder (2).

17. The examination device according to claim 16, wherein the operating connection comprises a wired or wireless mode connection and/or the reader (3) is configured to communicate with the identification element via a connection other than the operating connection.

18. An examination platform, comprising the device according to claim 16, and at least one probe equipped with an identification element.

19. A method for identifying a target probe of a plurality of probes collaborating with an examination device (500), comprising the following steps:
a communication step in which a reader (3) associated with a probe-holder (2) for supporting the target probe (1) communicates with an identification element associated with the target probe (1), and
a signalling step in which a signalling element emits a signal to a user as a function of the communication between the reader (3) and the identification element.

20. The method according to claim 19,
wherein the signal is suitable for indicating to the user a status of the target probe (1), comprising at least one of the following signal types:
a signal type which indicates in which probe-holder (2) of a plurality of probe-holders of the device the target probe (1) is placed,
a signal type for indicating that the probe is suitable for a predetermined examination or a given phase of an examination,
a signal type for indicating that the target probe (1) is undergoing maintenance,
a signal type for indicating that the probe is out of order,
a signal type for indicating that the target probe (1) is incorrectly inserted into the probe-holder (2),
a signal type for indicating that the target probe (1) is not connected to the examination device, and
a signal type for indicating that the target probe (1) is in test mode.

21. The method according to claim 19,
wherein prior to the use of the target probe (1) in a predefined examination the signalling element emits a first signal type which indicates in which probe-holder (2) the target probe (1) is placed at a first signalling level, and
during use and/or at the end of use, the signalling element emits a second signal type which indicates which probe-holder(s) is/are suitable for receiving the target probe (1) after use.

22. The method according to claim 19, further comprising the steps:
equipping each probe with an identification element,
equipping the examination device with probe-holders each comprising a reader (3) and/or a signalling element, or each comprising a central reader and/or a central signalling element, and/or equipping existing probe-holders of the device with readers or a central reader and/or signalling elements or a central signalling element,
recording in a data memory which probe-holder (2) is suitable for receiving which category of probe, and
optionally signalling to the user via the signalling element, if a probe is placed in a probe-holder (2) which is not suitable for this probe.

23. An examination method, comprising:
identifying a target probe of a plurality of probes in accordance with the method of claim 19,
acquiring examination data via the target probe (1) which is a first target probe,
determining via a processor of the device the recommendation for acquiring additional examination data based on the examination data acquired by the first target probe, and
identifying at least one second target probe suitable for acquiring additional examination data and signalling to the user in which probe-holder (2) the second probe is placed.

24. The method according to claim 23, wherein the recommendation for acquiring additional examination data is determined by an intelligent learning algorithm, which uses the examination data acquired by the first target probe as input data and determines as output data identification of a possible second target probe and/or identification of the probe-holder (2) comprising the second target probe.
